(19) United States
(12) Patent Application Publication
(10) Pub. No.: US 2024/0342411 A1
(43) Pub. Date: Oct. 17, 2024

(54) GAS SENSOR MODULE WITH ENHANCED FLOW

(71) Applicant: Mallinckrodt Pharmaceuticals Ireland Limited, Dublin (IE)

(72) Inventor: Peter J. Clark, Aylesbury (GB)

(21) Appl. No.: 18/630,745

(22) Filed: Apr. 9, 2024

Related U.S. Application Data
(60) Provisional application No. 63/458,990, filed on Apr. 13, 2023.

(51) Int. Cl. A61M 16/00; A61M 16/08

(52) U.S. Cl. CPC A61M 16/0003; A61M 16/0841; A61M 2016/0039

(57) ABSTRACT

A gas sensor module is provided for a therapeutic gas delivery device. The gas sensor module includes a sample inlet and a sample chamber forming a flow passage fluidly connected with the sample inlet and operable to receive sample gas, where the sample chamber includes a plurality of sensor chambers in communication with one or more corresponding sensors operable to measure at least one property of the sample gas. The flow passage directs a flow of the sample gas through the gas sensor module and includes one or more turns prior to each of the plurality of sensor chambers, each turn having an angle operable to promote mixing of the sample gas and promote diffusion of the sample gas onto each of the plurality of sensors.

### CLAIM OF PRIORITY

This application claims the benefit of U.S. Provisional Application No. 63/458,990, filed on Apr. 13, 2023, the entire contents of which are herein incorporated by reference in its entirety.

### FIELD

The present disclosure relates generally to gas sensor modules with enhanced flow. In one example, the present disclosure relates to gas sensor modules with enhanced flow for efficient gas flow over sensors in therapeutic gas delivery devices.

### BACKGROUND

A current method of sensing medical therapy gasses in a patent line is to tee off and take a small sample of the gas mix and deliver it to a sensor cluster that communicates with the main device delivery management system. The most important requirement is for the gas to be delivered in the most homogenous condition in a timely manner. The gasses can change as they are transported through the system where, for example, the oxygen molecules can combine with nitric oxide to form nitrogen dioxide, making a timely delivery time to the sensor bank an important requirement for accuracy of gas mix being delivered to the patient. It is also desired to limit the volume of gas within the sample line to ensure real time monitoring of the therapeutic mix being delivered directly to the patient.

### SUMMARY

In some aspects, the disclosure provides for a gas sensor module for a therapeutic gas delivery device. The gas sensor module may include a sample inlet operable to receive a sample gas comprising a mixture of a breathing gas and a therapeutic gas from a sample line connected to an inspiratory line of the therapeutic gas delivery device and a sample chamber forming a flow passage fluidly connected with the sample inlet and operable to receive the sample gas, the sample chamber includes a plurality of sensor chambers operable to be in communication with one or more corresponding sensors operable to measure at least one property of the sample gas. The flow passage may be operable to direct a flow of the sample gas from the sample inlet to the plurality of sensor chambers, between each of the plurality of sensor chambers, and from the plurality of sensor chambers to an outlet. The flow passage may include one or more turns prior to each of the plurality of sensor chambers, each turn having an angle operable to promote mixing of the sample gas and promote diffusion of the sample gas onto each of the one or more corresponding sensors.

In an aspect, the mixing of the sample gas is such that a concentration of the breathing gas and the therapeutic gas in the sample gas flowing over the plurality of sensor chambers is substantially the same as the concentration of the breathing gas and the therapeutic gas in the inspiratory line.

In some aspects, the flow passage reduces or prevents rotating eddies and dead zones in the sample gas flow through the sample chamber. In additional aspects, the flow passage creates a low back pressure or resistance to flow through the sample chamber. The mixing of the sample gas may provide a turbulent delivery of the sample gas to a surface of each of the one or more corresponding sensors. In some aspects, a volume of sample gas in the sample chamber is less than a volume of sample gas that would be needed in a sample chamber without mixing.

In some aspects, the angle of the one or more turns is about 90 degrees. The one or more turns are operable to change direction of the flow passage within the sample chamber.

In an aspect, the sample chamber includes an inner housing having an upper portion, a lower portion, a vertical axis, and a horizontal axis transverse to the vertical axis. At least one of the one or more turns is along the vertical axis and/or the horizontal axis. The one or more turns are about 90 degrees along the vertical axis and/or the horizontal axis of the sample chamber. In some aspects, at least one of the plurality of sensor chambers is located in the lower portion of the sample chamber and the sample inlet is located in the upper portion of the sample chamber.

The plurality of sensor chambers may include a first sensor chamber that has a first sensor in the lower portion of the sample chamber, and the flow passage may include about one to about four turns, each having angles of about 90 degrees, between the sample inlet in the upper portion of the sample chamber and an inlet of the first sensor chamber. In some aspects, the flow passage between the sample inlet and the first sensor chamber may include a first vertical turn along the vertical axis, a second vertical turn along the horizontal axis, a first horizontal turn along the horizontal axis subsequent to the second turn, and a third vertical turn along the vertical axis. In an aspect, the first sensor chamber may be a nitrogen dioxide sensor chamber and the first sensor may be a nitrogen dioxide sensor.

In some aspects, the plurality of sensor chambers may include a second sensor chamber comprising a second sensor in the lower portion of the sample chamber, and the flow passage may include about one to about four turns, each having angles of about 90 degrees, between an outlet of the first sensor chamber and an inlet of the second sensor chamber. The flow passage between the outlet of the first sensor chamber and the inlet of the second sensor chamber may include a fourth vertical turn along the horizontal axis and a fifth vertical turn along the vertical axis subsequent to the fourth vertical turn. The second sensor chamber may be a nitric oxide sensor chamber and the second sensor is a nitric oxide sensor.

In some aspects, the plurality of sensor chambers includes a third sensor chamber comprising a third sensor in the upper portion of the sample chamber, and wherein the flow passage includes about one to about four turns, each having angles of about 90 degrees, between an outlet of the second sensor chamber and an opening of the third sensor chamber. The flow passage between the outlet of the second sensor chamber and opening of the third sensor chamber may include a sixth vertical turn along the horizontal axis, a second horizontal turn along the horizontal axis, and a seventh vertical turn along the vertical axis subsequent to the second horizontal turn. The third sensor chamber may be a humidity and/or temperature sensor chamber and third sensor may be a humidity and/or temperature sensor.

In some aspects, the flow passage directs the sample gas directly into the opening of the humidity and/or temperature sensor chamber such that the sample gas exits the humidity and/or temperature sensor chamber through the opening. The flow passage directs the sample gas directly into the opening from a vertical portion along a vertical axis of the sample chamber.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Implementations of the present technology will now be described, by way of example only, with reference to the attached figures, wherein:

FIG. 1A is an exploded view of an exemplary gas sensor module.

FIG. 1B is an exploded view of an exemplary gas sensor module.

FIG. 1C is an isometric view of an exemplary sensor chamber with the cover removed.

FIG. 2A is an isometric view of the back of an exemplary gas sensor module.

FIG. 2B is a side view of an exemplary gas sensor module.

FIG. 2C is a front isometric view of an exemplary gas sensor module with the cover removed.

FIG. 2D is a top view of an exemplary gas sensor module with the cover removed.

FIGS. 3A-3C show models of gas flow through an exemplary gas sensor module. FIG. 3A shows a top view of the gas flow through an exemplary gas sensor module. FIG. 3B shows a side view of the gas flow through an exemplary gas sensor module. FIG. 3C shows a side view of the gas flow through the outlet of an exemplary gas sensor module.

FIGS. 4A-4D show models of gas flow through another exemplary gas sensor module. FIG. 4A shows an isometric view of gas flow through an exemplary gas sensor module. FIG. 4B shows a top view of gas flow through an exemplary gas sensor module. FIG. 4C shows a side view of gas flow through an exemplary gas sensor module. FIG. 4D shows a close-up view of gas flow through an exemplary gas sensor module.

FIG. 5 shows a model of flow velocity through an exemplary gas sensor module.

FIG. 6A shows an isometric cutaway view of the flow passage of an exemplary gas sensor module.

FIG. 6B shows an isometric see-through view of the flow passage of an exemplary gas sensor module.

FIG. 7 shows a gas sensor module installed in a therapeutic gas delivery device in one example.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout the above disclosure will now be presented. The term “coupled” is defined as connected, whether directly or indirectly through intervening components, and is not necessarily limited to physical connections. The connection can be such that the objects are permanently connected or releasably connected.

The term “substantially” is defined to be essentially conforming to the particular dimension, shape or other word that substantially modifies, such that the component need not be exact. For example, “substantially cylindrical” means that the object resembles a cylinder, but can have one or more deviations from a true cylinder. In another example, “substantially homogeneous” may mean greater than 75% homogeneous, greater than 80% homogeneous, or greater than 90% homogeneous.

The terms “comprising,” “including” and “having” are used interchangeably in this disclosure. The terms “comprising,” “including” and “having” mean to include, but not necessarily be limited to the things so described.

Generally, the ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc. Other than in the operating examples, or where otherwise indicated, all numbers expressing quantities of ingredients and/or reaction conditions may be modified in all instances by the term “about,” meaning within +/−5% of the indicated number.

Disclosed herein is an enhanced flow gas sensor module with a sample line having a flow passage within a sample chamber, where the flow passage has one or more turns between a plurality of sensors for measuring at least one property of a sample gas in a therapeutic gas delivery device. The sample gas may be a mixture of a breathing gas and a therapeutic gas being delivered to a patient through an inspiratory line of the therapeutic gas delivery device. The enhanced flow gas sensor module improves previous known gas delivery systems by ensuring sample gas volumes are minimized and sample gas mixing is enhanced for accurate measurement of concentrations. For example, the enhanced flow gas sensor module maximizes gas flow across a sensor face and promotes enhanced flow to ensure the sample gas mixture is even to prevent rotating eddies and dead zones in the gas flow. The enhanced flow gas sensor module is designed to include several twists and turns as the flow of sample gas passes through the sensor zones/chambers and eliminates the possibility of any percentage of sample gas bypassing the sensor. For example, the enhanced flow gas sensor module provides for mixing of the sample gas such that the sample gas mixture over one or more sensors in the enhanced flow gas sensor module is homogeneous. In some examples, the sample gas mixture over one or more sensors in the enhanced flow gas sensor module is substantially homogeneous. The sample gas is directed through the sensor unit through a passageway that is designed to create a low back pressure or resistance to flow whilst ensuring an enhanced delivery to the sensor surface—this limits the volume of gas in the sample line whilst delivering a sufficient volume of fresh sample gas to the sensors.

Conventionally, sensing medical therapy gases, such as nitric oxide, in a patient line is done by taking a small sample of the gas mixture from a sample tee and delivering it to a sensor cluster that communicates with a main device delivery management system. The most important requirement is for the gas to be delivered in the most homogenous condition in a timely manner. The gases can change as they are transported through the system where, for example, oxygen molecules can combine with nitric oxide to form nitrogen dioxide, making a timely delivery to the sensor cluster an important requirement for accuracy of the gas mixture being delivered to the patient. Thus, the sensing of a sample gas mixture in a therapeutic gas delivery device may not timely or accurately represent the mixture being delivered to the patient, which can interfere with the effective treatment of the patient.

The enhanced flow gas sensor module described herein overcomes the limitations of the conventional gas sensors. An advantage of the enhanced flow gas sensor module is that it delivers a quantity of sample gas to a plurality of sensor chambers that will ensure the sensors can accurately detect concentrations whilst minimizing the volume removed from the patient line. It is an advantage to limit the volume of gas within the sample line to ensure real time monitoring of the therapeutic mix being delivered directly to the patient. It is also an advantage to deliver the gas in as close as possible to the same concentration mix as is being delivered to the patent.

In some examples, the gas sensor module includes a flow passage that has one or more turns between a plurality of sensors for measuring at least one property of a sample gas in a therapeutic gas delivery device. The one or more turns may be operable to change the direction of the flow passage within the sample chamber. The mixing of the sample gas may be such that the concentration of the breathing gas and the therapeutic gas in the sample gas flowing over a plurality of sensor chambers is substantially the same as the concentration of the breathing gas and the therapeutic gas in the inspiratory line.

The angle of each of the one or more turns may be operable to promote mixing of the sample gas and promote diffusion of the sample gas onto each of the plurality of sensors. In some aspects, the angle of each of the one or more turns may be between about 0 and about 90 degrees. In some additional aspects, the angle of the one or more turns may be about 0 degrees, about 15 degrees, about 30 degrees, about 45 degrees, about 60 degrees, about 75 degrees, or about 90 degrees.

The flow passage may reduce or prevent rotating eddies and dead zones in the sample gas flow through the sample chamber. In some embodiments, the flow passage may create a low back pressure or resistance to flow through the sample chamber. The mixing of the sample gas, due to the design of the flow passage, may provide turbulent delivery of the sample gas to a surface of each of the one or more sensors. In some embodiments, the volume of sample gas in the sample chamber is less than the volume of sample gas that would otherwise be needed in the sample chamber without the mixing caused by the configuration of the flow passage and the turbulent flow states caused by the flow passage. In some examples, the flow passage can be a tubular flow passage.

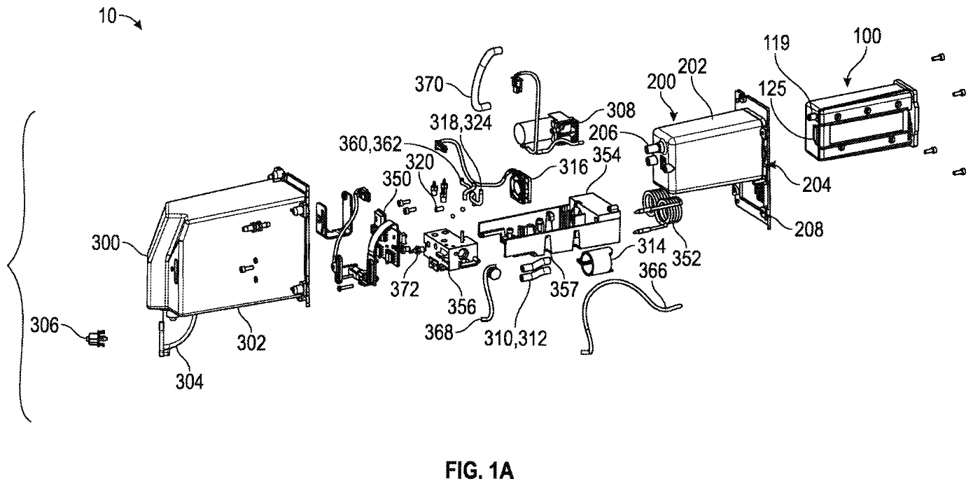

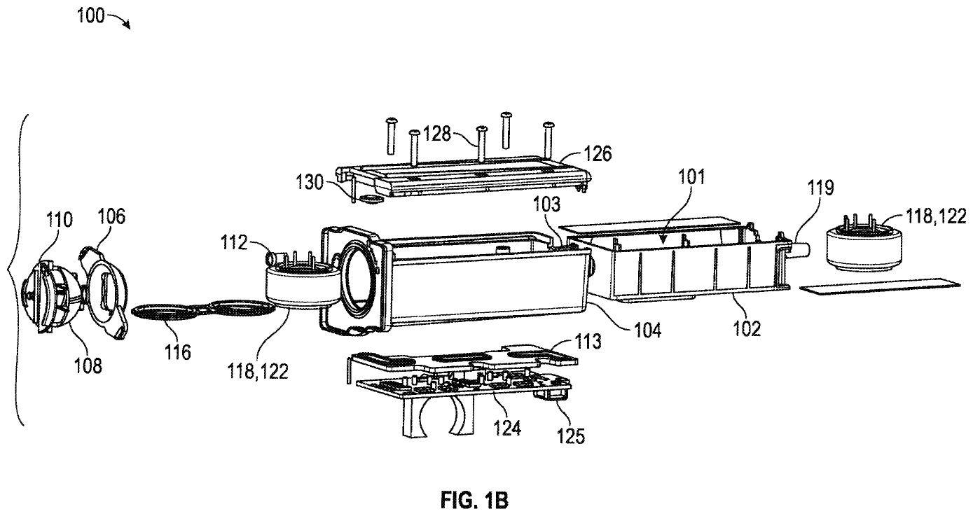

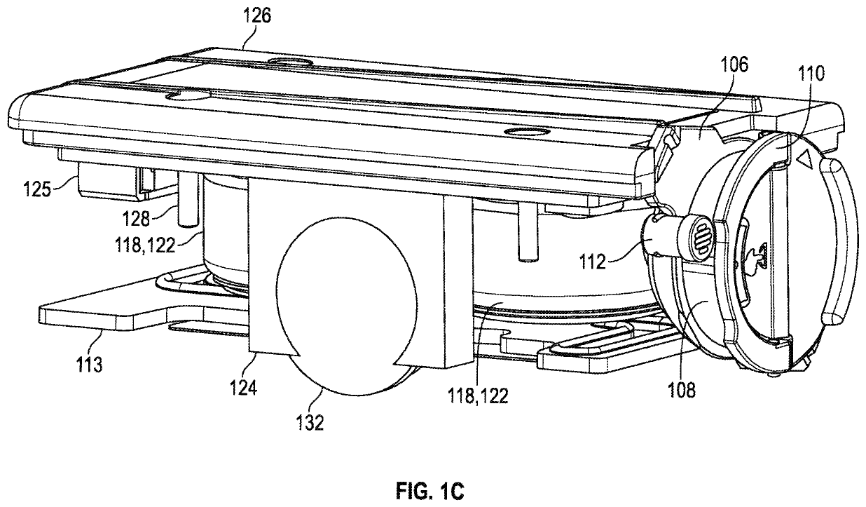

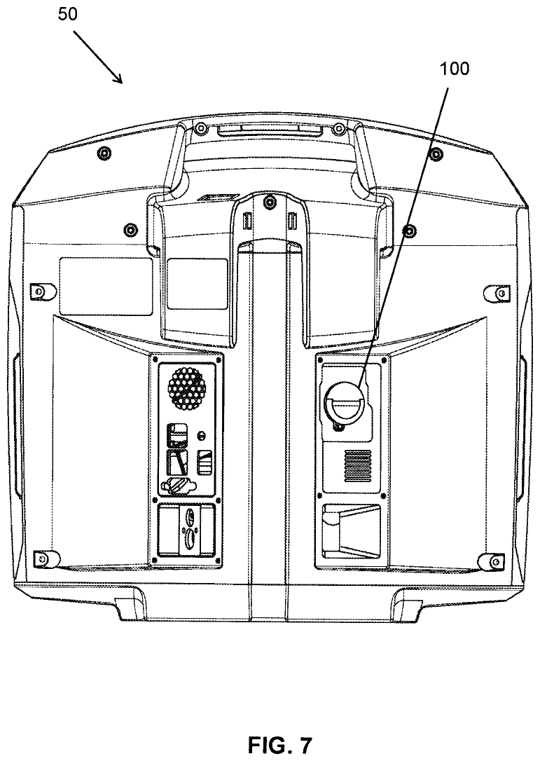

The enhanced flow gas sensor module can be utilized in an exemplary gas sensor assembly shown, for example, in FIGS. 1A and 1B, which show exploded views of an exemplary gas sensor assembly, and in FIG. 1C, which shows an isometric back view of an exemplary sensor chamber with the cover removed. The gas sensor assembly 10 includes a gas sensor module 100 and an assembly inner housing 200 operable to removably receive the gas sensor module 100. The assembly inner housing 200 includes a module receiving portion 202 which forms a module receiving recess 204. The gas sensor module 100 is removably received in the module receiving recess 204. As such, the gas sensor module 100 is removably coupled with the assembly inner housing 200. The gas sensor assembly 10 can also include a gas analyzer unit 300 with an assembly main housing 302 operable to receive the assembly inner housing 200. In some examples, the assembly inner housing 200 is removably coupled with the assembly main housing 302. In other examples, the assembly inner housing 200 is fixedly coupled with the assembly main housing 302. The gas analyzer unit 300 is contained within a therapeutic gas delivery device 50. In at least one example, the assembly main housing 302 is coupled with and in fluid communication with the therapeutic gas delivery device 50. In some examples, the gas sensor module 100 is nested within the assembly inner housing 200, which is nested within the assembly main housing 302, such that the gas sensor module 100 is coupled with and in fluid communication with the therapeutic gas delivery device 50. In other examples, the assembly inner housing 200 and the gas analyzer unit 300 can be integrated as a single unit operable to receive the gas sensor module 100. In additional examples, the therapeutic gas delivery device 50 is operable to receive the gas sensor module 100. FIG. 7 illustrates the gas sensor module 100 installed into the therapeutic gas delivery device 50.

The therapeutic gas delivery device 50 is operable to deliver therapeutic gas to a patient. For example, the therapeutic gas delivery device 50 can deliver therapeutic nitric oxide (NO) gas to a patient. The gas sensor module 100, the assembly inner housing 200, and the assembly main housing 302 are positioned such that gas can flow from a breathing circuit of the therapeutic gas delivery device 50, through a sample tube, through the gas analyzer unit 300, through the assembly inner housing 200, to the gas sensor module 100. In at least one example, a sample tube can be fluidly connected to a breathing circuit of the gas delivery device 50 and the gas sensor module 100 is operable to receive the sample gas from the sample tube. In at least one example, the breathing circuit of the therapeutic gas delivery device 50 includes a sample tee which is operable to receive the sample tube such that at least a portion of the gas in the breathing circuit flows through the sample tube. Additionally, in at least one example, the assembly inner housing 200 can include a port 206 which can be fluidly connected with a port 304 on the gas analyzer unit 300, which can be fluidly connected with the sample tube. The port 206 can receive the sample gas from the therapeutic gas delivery device 50, through the gas analyzer unit 300 port 304 and provide the sample gas to the gas sensor module 100.

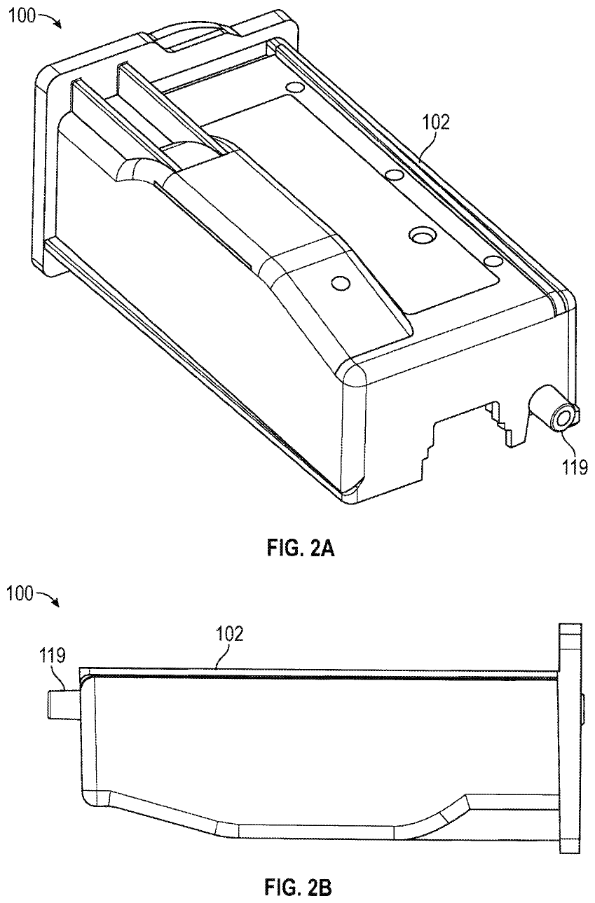

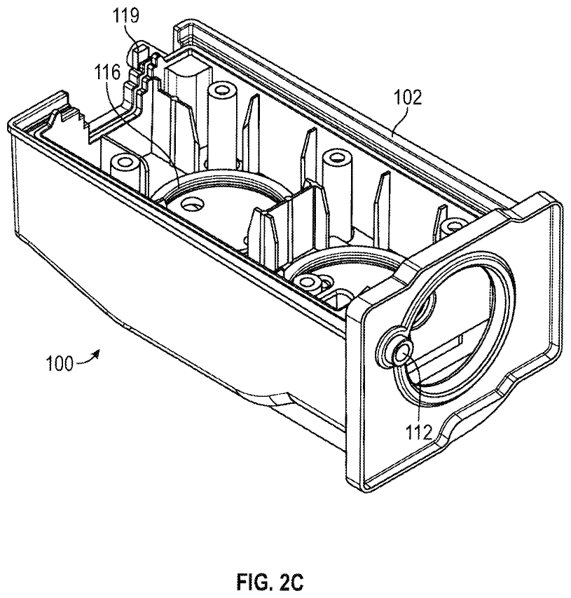

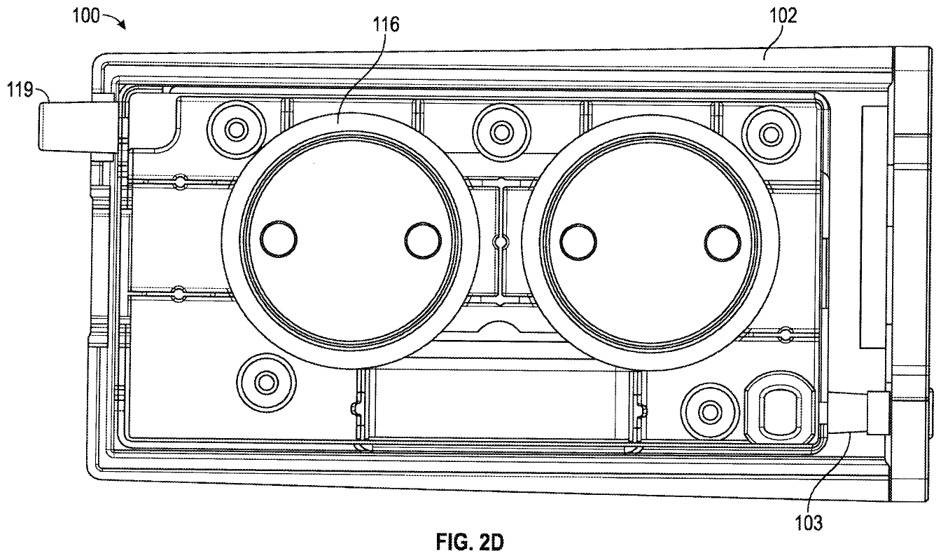

FIGS. 2A-2D illustrate the gas sensor module 100 from a back isometric view (FIG. 2A), a side view (FIG. 2B), a front isometric view with the cover removed (FIG. 2C), and a top view with the cover removed (FIG. 2D). FIGS. 1A and 1B illustrate exploded views of the gas sensor module 100. The gas sensor module 100 includes a sample chamber 101. The sample chamber 101 receives the sample gas from the therapeutic gas delivery device 50. The sample chamber 101 is fluidly connected with an inlet 119. The inlet 119 is fluidly connected with a therapeutic gas delivery device and is operable to receive the sample gas. In some examples, the inlet 119 is fluidly connected to the port 206 of the assembly inner housing 200, which is fluidly connected to the port 304 of the gas analyzer unit 300, which is fluidly connected with the sample tube in the therapeutic gas delivery device 50. In at least one example, the sample chamber 101 is operable to receive the sample gas from the therapeutic gas delivery device 50. The sample chamber 101 can include an inner housing 102. The inner housing 102 can include an outlet 103 through which the sample gas can be removed from the sample chamber 101. The outlet 103 can be, for example, an opening formed in the inner housing 102. In at least one example, the gas sensor module 100 includes an outer housing 104 which at least partially surrounds the inner housing 102. In some examples, the outer housing 104 can include at least one of the following: a cam element 106, a cam spindle 108, a handle 110, a handle axle 114, a vent cap 112, and/or a gasket 113. In at least some examples, the cam element 106, the cam spindle 108, the handle 110, and/or the handle axle 114 can be used to facilitate ease of insertion/removal of the gas sensor module 100 by the user via a locking/unlocking action of the vent cap 112. In some examples, the handle 110 can be a flip-up pull tab, as seen in FIG. 1B. The gasket 113 can help to prevent leaks from the pneumatic circuit (e.g., the pressurized sample gas circuit/flow passage), stopping sample gas from interacting with the electronics. In at least one example, the gasket 113 can be made of silicon rubber. The gasket 113 can be made of other types of materials configured to prevent leaks.

In some embodiments, the inner housing 102 can include an upper portion, a lower portion, a vertical axis, and a horizontal axis transverse to the vertical axis. At least one of the one or more turns of the flow passage 144 may be along the vertical axis and/or the horizontal axis. In some embodiments, the one or more turns are about 90 degrees along the vertical axis and/or the horizontal axis of the sample chamber 101.

The gas sensor module 100 includes a plurality of sensors 118. The plurality of sensors 118 is operable to be in communication with a plurality of corresponding sensor chambers 140, which are operable to receive the sample gas. Each sensor chamber 140 may include an inlet or an opening to the sensor chamber and an outlet from the sensor chamber. The flow passage 144 is operable to direct a flow of the sample gas from the inlet 119 to the plurality of sensor chambers 140, between each of the plurality of sensor chambers 140, and from the plurality of sensor chambers to the outlet 103. In some embodiments, at least one of the plurality of sensor chambers 140 may be in the lower portion of the sample chamber 101. In some additional embodiments, the inlet 119 may be located in the upper portion of the sample chamber 101. The sensors 118 are operable to measure at least one property of the sample gas. For example, the sensors 118 can include two or more of gas detection sensors, humidity sensors, and/or temperature sensors.

In at least one example, the gas sensor module 100 can include two or more gas detection sensors 122. In at least one example, the gas sensor module 100 can include two or more different sensors 118. As illustrated in FIGS. 1B and 1C, the gas sensor module 100 can include a humidity sensor (not shown) and two gas detection sensors 122. In other examples, the gas sensor module 100 can include one or more gas detection sensors 122 and a humidity sensor. The gas detection sensors 122 can include one or more of an NO sensor, an NO₂ sensor, an O₂ sensor, or combinations thereof. In at least one example, the gas detection sensors 122 can include an NO sensor and an NO₂ sensor. Each gas detection sensor may be operable to be in communication with a sensor chamber 140. The plurality of sensor chambers 140 may include NO sensor chambers, NO₂ sensor chambers, O₂ sensor chambers, humidity sensor chambers, temperature sensor chambers, or combinations thereof. The sensor chambers may include one or more of an NO sensor, an NO₂ sensor, an O₂ sensor, a humidity sensor, temperature sensor, or combinations thereof. While FIG. 1B illustrates two gas detection sensors 122, one, three, or more gas detection sensors 122 can be included. The property of the sample gas being measured can be one or more of a concentration of NO, a concentration of NO₂, a concentration of O₂, humidity, temperature, or a combination thereof. As illustrated in FIG. 1B, the gas sensor module 100 includes a sensor seal 116 coupled with at least one of the sensors 118. As illustrated in FIG. 1B, the gas sensor module 100 can include a humidity sensor seal 130 operable to be coupled with a humidity sensor (not shown) that can be integrated with the sensing circuit 124. For example, the humidity sensor can be located near the outlet 103 of the sample chamber 101.

In some examples, a humidity sensor and/or temperature sensor can be located in an upper portion (e.g., near the outlet 103 and/or inlet 119) of the gas sensor module 100 along the flow passage 144. In some examples, one or more gas detection sensors 122 can be located in a bottom portion of the gas sensor module 100 along the flow passage 144.

In some examples, the gas sensor module 100 can have a height of about less than 5 inches. In some examples, the gas sensor module 100 can have a height of about less than 0.5 inches, about 0.5 inches to about 1 inch, about 1 inch to about 1.5 inches, about 1.5 inches to about 2 inches, about 2 inches to about 2.5 inches, about 2.5 inches to about 3 inches, about 3 inches to about 3.5 inches, about 3.5 inches to about 4 inches, about 4 inches to about 4.5 inches, about 4.5 inches to about 5 inches, or more. In some examples, the gas sensor module 100 can have a length (e.g., from the inlet 119 end to the outlet 103 end) of about less than 5 inches. In some examples, the gas sensor module 100 can have a length of less than about 0.5 inches, about 0.5 inches to about 1 inch, about 1 inch to about 1.5 inches, about 1.5 inches to about 2 inches, about 2 inches to about 2.5 inches, about 2.5 inches to about 3 inches, about 3 inches to about 3.5 inches, about 3.5 inches to about 4 inches, about 4 inches to about 4.5 inches, about 4.5 inches to about 5 inches, about 5 inches to about 5.5 inches, about 5.5 inches to about 6 inches, or more. In some examples, the gas sensor module 100 can have a width of about less than 5 inches. In some examples, the gas sensor module 100 can have a width of about 0.5 inches to about 1 inch, about 1 inch to about 1.5 inches, about 1.5 inches to about 2 inches, about 2 inches to about 2.5 inches, about 2.5 inches to about 3 inches, about 3 inches to about 3.5 inches, about 3.5 inches to about 4 inches, about 4.5 inches to about 5 inches, about 5 inches to about 5.5 inches, about 5.5 inches to about 6 inches, or more.

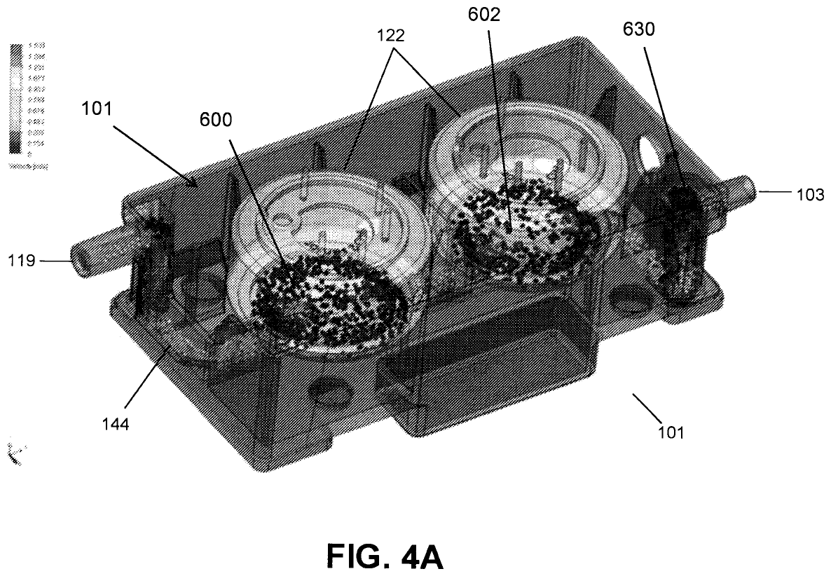

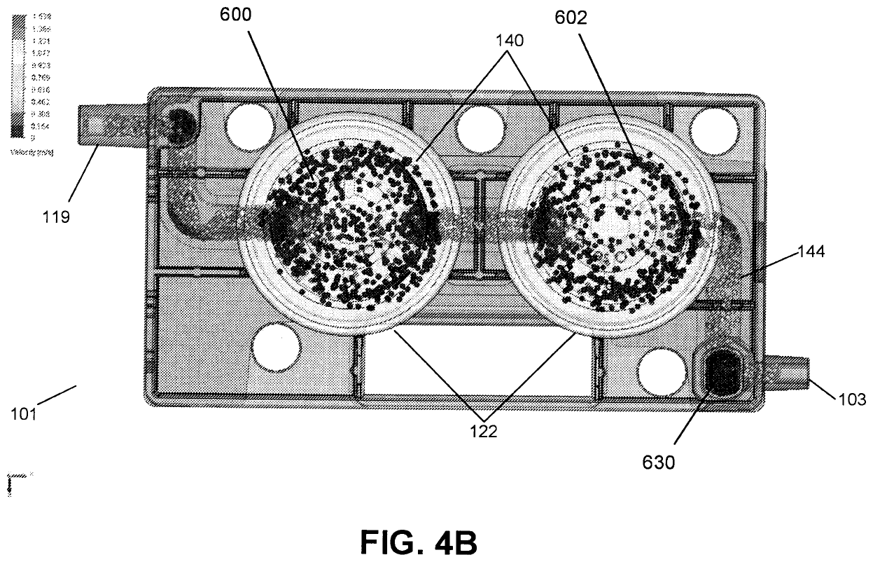

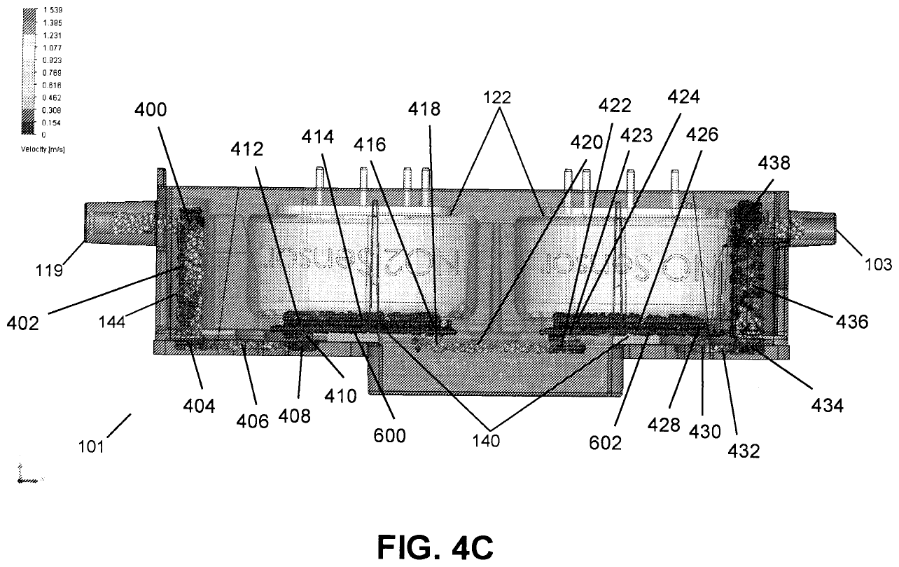

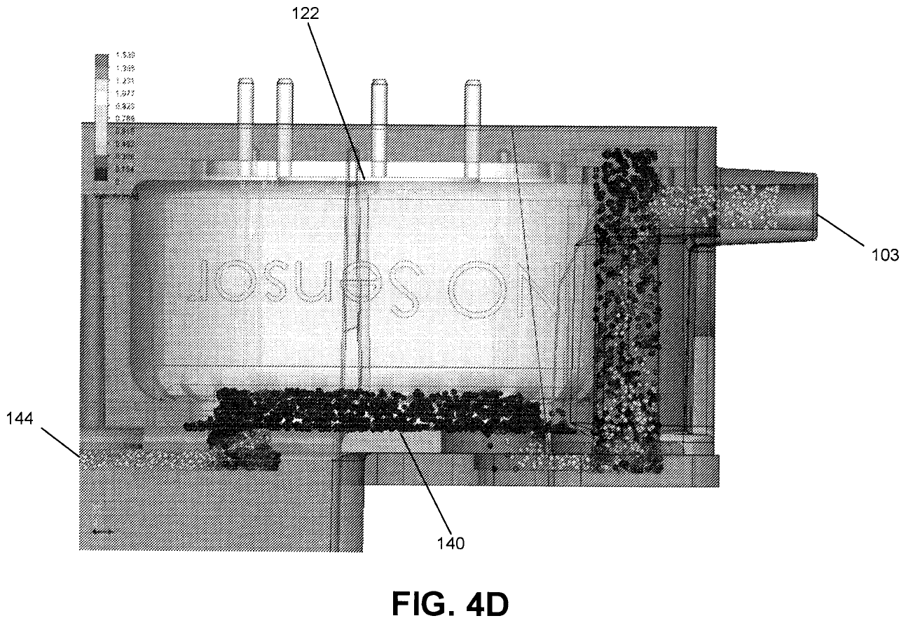

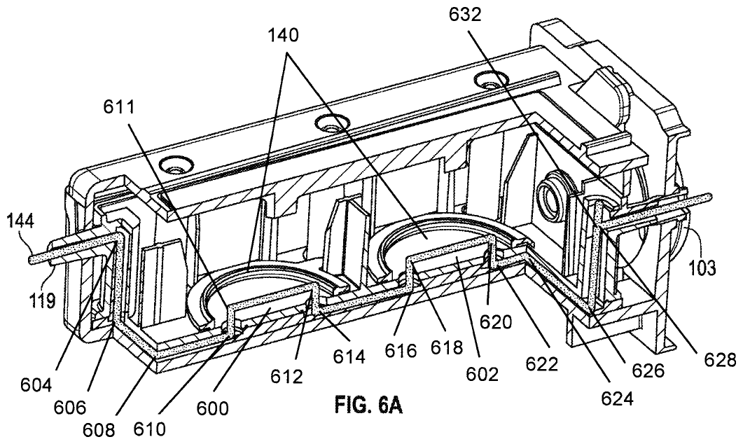

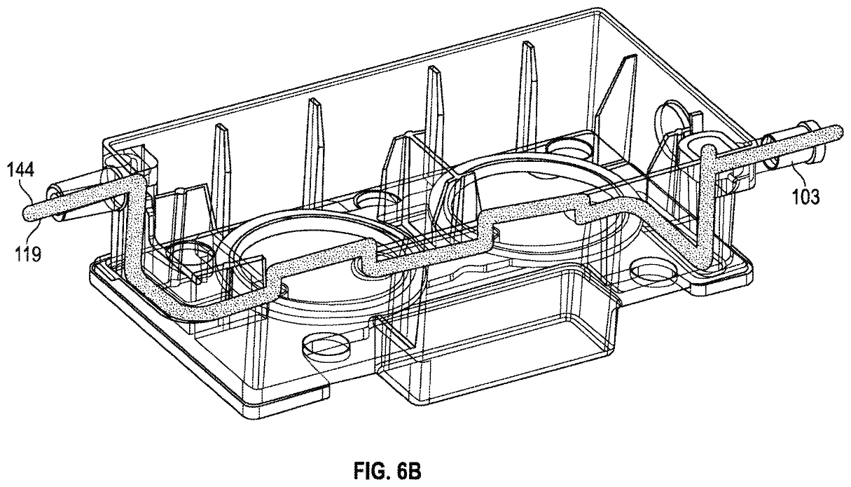

In an exemplary embodiment (see FIGS. 6A-6B), the plurality of sensor chambers 140 includes a first sensor chamber 600 comprising a first sensor 118, 122 in the lower portion of the sample chamber 101 and the flow passage 144 includes about one to about four turns, each having angles of about 90 degrees, between the inlet 119 in the upper portion of the sample chamber 101 and an inlet of the first sensor chamber 600. The flow passage 144 between the inlet 119 and the first sensor chamber 600 includes a first vertical turn along the vertical axis, a second vertical turn along the horizontal axis, a first horizontal turn along the horizontal axis, and a third vertical turn along the vertical axis subsequent to the first horizontal turn. The first sensor chamber 600 may be NO₂ sensor chamber and the first sensor 118, 122 may be a NO₂ sensor. The plurality of sensor chambers 140 may include a second sensor chamber 602 comprising a second sensor 118, 122 in the lower portion of the sample chamber 101. The flow passage 144 may include about two turns, each having angles of about 90 degrees, between an outlet of the first sensor chamber and the inlet of the second sensor chamber. The flow passage 144 between the outlet of the first sensor chamber 600 and the inlet of the second sensor chamber 602 may include a first vertical turn along the vertical axis and a second vertical turn along the vertical axis subsequent to the first vertical turn. The second sensor chamber 602 may be a NO sensor chamber and the second sensor 118, 122 may be a NO sensor. The plurality of sensor chambers 140 may include a third sensor chamber 630 and a third sensor (e.g., humidity sensor and/or temperature sensor) in the upper portion of the sample chamber 101, as illustrated, for example, in FIG. 4A. The flow passage 144 may include about one to about four turns, each having angles of about 90 degrees, between the outlet of the second sensor chamber 602 and the opening of the third sensor chamber 630. The flow passage 144 between the outlet of the second sensor chamber 602 and the opening of the third sensor chamber 630 may include a first vertical turn along the horizontal axis, a first horizontal turn along the horizontal axis, and a second vertical turn along the vertical axis subsequent to the first horizontal turn. The third sensor chamber 630 may be a humidity and/or a temperature sensor chamber, and the third sensor may be a humidity and/or a temperature sensor. The flow passage 144 may direct the sample gas directly into the opening of the humidity and/or temperature sensor chamber (e.g., third sensor chamber 630) and the sample gas may exit the humidity and/or temperature sensor chamber through the same opening. The flow passage 144 may direct the sample gas directly into the opening along the vertical axis of the sample chamber 101.

Returning to FIGS. 1A-1B, the gas sensor module 100 includes a sensing circuit 124 that can be coupled with the sensors 118. The sensing circuit 124 is operable to detect and report the measured properties of the sample gas from the sensors 118. The sensing circuit 124 can be communicatively coupled with the gas delivery device 50. In an example, the sensing circuit 124 can be operable to report measured properties of the sample gas to a gas analyzer controller 350 in the gas analyzer unit 300. In an example, the gas analyzer controller 350 can be operable to report measured properties of the sample gas to the therapeutic gas delivery device 50. The sensing circuit 124 can be coupled with the gas analyzer controller 350 and/or the gas delivery device 50 by any suitable wired or wireless connection, for example Ethernet, Bluetooth, RFID, or fiber optic cable. In at least one example, the sensing circuit 124 and/or the gas analyzer controller 350 can be operable to store measured properties of the sample gas. The gas sensor module 100, by the sensing circuit 124, can be operable to electronically retain serial numbers, calibration data, and/or usage information of the gas sensor module 100. In another example, the gas analyzer controller 350 can be operable to electronically retain serial numbers, calibration data, and/or usage information of the gas sensor module 100. Thereby, components can continue to be tracked and traced even when the gas sensor module 100 is disconnected from the gas delivery device 50. The sensing circuit 124 can include a connector 125 operable to connect the sensing circuit 124 of the gas sensor module 100 with the gas analyzer controller 350, and thus, the gas delivery device 50. Accordingly, the gas sensor module 100 can be hot swapped, and the connector 125 is easily connected with the gas delivery device 50 without additional expertise or tools.

The gas sensor module 100 additionally includes a cover 126, which can be coupled with the outer housing 104. In at least one example, the cover 126 can be removably coupled with the outer housing 104 by fasteners 128. Fasteners 128 can be, for example, at least one of: screws, nails, nuts and bolts, hook and loop fasteners, adhesives, and/or any other suitable fasteners.

The gas sensor module 100 is self-contained within the therapeutic gas delivery device 50 and is swappable with another gas sensor module 100. The containment of all of the sensors and/or analysis elements for the gas sample provides for the ability of a hot-swap in the event of a need for recalibration, component failure, and/or contamination. For example, the gas sensor module 100 can be replaced in the event of gas sensor module 100 failure, sample line filter failure, and/or when the service period for the calibration of the gas sensor module 100 is due to expire. Additionally, the modularization of the gas sensor module 100 simplifies the future addition of sensors 118 for analytes such as O₂ or volatile organic compounds (VOCs) without the need to modify the overall gas delivery device 50, instead “upgrading” to a next-generation gas sensor module. A replacement gas sensor module 100 can simply be installed and the gas delivery device 50 can then immediately be put back into service. The gas sensor module 100 can be replaced with a pre-calibrated gas sensor module 100 by a responsible person in a matter of minutes without the need for special tools or equipment. For example, the replacement of the gas sensor module 100 can result in less than five minutes of down time in the measurement of at least one property of the sample gas. In at least one example, the replacement of the gas sensor module 100 can result in less than three minutes of down time in the measurement of at least one property of the sample gas. In some examples, the replacement of the gas sensor module 100 can result in less than one minute of down time in the measurement of at least one property of the sample gas.

In another example, the replacement of the gas sensor module 100 can result in no down time in delivery of therapeutic gas from the therapeutic gas delivery device 50. In this example, the delivery of therapeutic gas to the patient is uninterrupted by the replacement of the gas sensor module 100 because the gas sensor module 100 analyzes sample gas, separate from the therapeutic gas in the breathing circuit. In addition, because the gas sensor module 100 is self-contained, it does not require shutdown of the therapeutic gas delivery device 50 or any stoppage in flow of therapeutic gas to the patient. This allows for the therapeutic gas delivery device 50 to continuously deliver therapeutic gas to the patient through the breathing circuit while the gas sensor module 100 is swapped for a new pre-calibrated gas sensor module 100. In at least one example, the therapeutic gas delivery device 50 can be continuously operable when the gas sensor module 100 is replaced. Additionally, sample detection by the gas sensor module 100 can begin approximately five minutes after installation, following completion of a low calibration protocol. In at least one example, the low calibration protocol can start automatically upon installation of a new gas sensor module 100. The hot-swap ability of the gas sensor module 100 has a significant, positive impact on user experience and device downtime. The gas sensor module 100 being pre-calibrated, or calibrated prior to installation, eliminates the need for onsite high calibration of NO sensors and/or other sensors and enables fast and simple replacement of a failed or expired gas sensor module, allowing for off-site re-calibration and repair if applicable.

The gas sensor module 100 can be utilized, or in-use, and maintain calibration stability for at least one month. In at least one example, the in-use calibration stability period for the gas sensor module 100 can be extended from the conventional one month to approximately three months. In at least one example, the gas sensor module 100 can have a shelf-life calibration stability period (for example, stability when not installed in a gas delivery device 50) of at least 1 month, alternately at least 3 months, alternately at least 6 months, or alternately at least 1 year. In some examples, the shelf-life of the gas sensor module 100 may be extended by including a battery 132, as illustrated in FIG. 1C, or other voltage source to provide an electrical potential across the sensors 118, 122 during storage to maintain the calibration. In at least one example, the gas sensor module 100 can include an expiration date. A user can be provided with gas sensor module replacement reminders/alarms via, for example, a graphic user interface and/or an app and/or program associated with the therapeutic gas delivery device.

In at least one example, the gas sensor module 100 can include and/or be electrically connected to an apparatus (not shown) which includes a voltage source that may be used in conjunction with an ultra-low power consumption setting to ensure the sensors 118 retain calibration stability for a predetermined period, for example, up to 6 months. The plurality of sensors 118 in the gas sensor module 100 can be pre-calibrated, and with the apparatus, an electrical potential can be provided across the sensors 118 to maintain the calibration of the sensors 118. For example, the voltage source may provide an electrical potential across the plurality of sensors 118 of the gas sensor module 100 at predetermined times to maintain calibration stability of the sensors 118 when the gas sensor module 100 is in a non-installed configuration. As such, the end-user may order multiple gas sensor modules 100, keeping them in storage until they are required to replace in-use gas sensor modules 100 when recalibration and/or replacement is due. In at least one example, the voltage source can be a battery or a power transformer. In at least one example, the voltage source can be a battery 132 internal to the gas sensor module 100, as seen in FIG. 1C. In other examples, the voltage source can be external to the gas sensor module 100. The voltage source can cease to provide electrical potential across the sensors 118 when the gas sensor module 100 is installed within the therapeutic gas delivery device 50. In at least one example, the apparatus and the voltage source can be removable from the gas sensor module prior to installation in the therapeutic gas delivery device 50. In another example, the voltage source can remain connected to the gas sensor module 100 after installation but no longer provide an electrical potential across the sensors 118, 122 of the gas sensor module 100. In at least one example, as illustrated in FIG. 1C, the battery 132 can directly connect with the sensing circuit 124 such that a separate apparatus is not needed to connect the battery 132 to the gas sensor module 100.

The implementation of pre-calibration and/or off-site calibration provides for calibration accuracy. For example, the conventional, single-point high calibration protocol assumes a single linear function across the range of administered NO concentrations. While sufficient to address current requirements for a +/−20% calibration accuracy, this could be improved upon significantly by employing a multi-point calibration protocol, something that is not compatible with a user-run calibration but which can be carried out automatically in a factory calibration scenario. With such an approach, calibration functions for multiple sub-ranges of NO concentration may be generated and stored for implementation (for example, in the form of a simple lookup table in device memory). The gas sensor module 100 can then determine the appropriate calibration function to use when measuring gas delivery based on, for example, the set dose and the range in which it sits. This is particularly important in pediatric or other low concentration applications for NO administration, where many calibration gases are supplied at a set concentration of 45 ppm, often more than twice the administered NO concentration. This would also address an issue experienced with certain users, who are uncomfortable with the display of a concentration that may be up to 20% less/greater than the set dose.

Furthermore, an off-site (for example factory) calibration and/or pre-calibration can utilize a calibration manifold 356 (shown in FIG. 1A) that can control at least one of temperature, relative humidity, and pressure, facilitating the generation of calibration functions that not only provide a more accurate measurement of gas, such as NO, in specific sub-ranges but also enable compensation for different temperatures, pressures, and relative humidity values.

Additionally, an off-site calibration and/or pre-calibration can facilitate precise measurement of the gas, such as NO, concentration used in the calibration gas mixture. Rather than use calibrated gas cylinders that have been prepared in batches for distribution to end-users, calibration gas can be precisely quantified in terms of gas concentration.

FIGS. 1A and 1B illustrate a detailed exploded view of a gas sensor assembly 10. As described herein, the gas sensor assembly 10 includes the gas sensor module 100 which is removably received in the assembly inner housing 200. The gas sensor module 100 can be removably coupled with the assembly inner housing 200 by one or more fasteners such as, for example, screws, clips, rotatable abutments, or any other suitable fastener such that the gas sensor module 100 can be removed from the assembly inner housing 200 without special tools or expertise. The assembly inner housing 200 can be received in and/or coupled with the assembly main housing 302, which is within or in fluid communication with the therapeutic gas delivery device. In an example, the assembly inner housing 200 and the assembly main housing 302 remain fixed within the therapeutic gas delivery device, while the gas sensor module 100 is removably replaced as needed.

A sample gas is taken from the therapeutic gas delivery device and passed to the gas sensor module 100 through the gas sensor assembly 10 such that the gas sensor module 100 can detect and report at least one property of the sample gas. The sample gas can enter the assembly main housing through port 304. In an example, a two stage filter luer interface 306 can be connected to the port 304, external to the assembly main housing 302. The port 304 can be fluidly connected to a pump 308 inside the assembly main housing 302. The pump 308 is operable to pump the sample gas through the gas sensor module 100. The pump 308 can retrieve the sample gas from the gas delivery device, for example, through the port 304 and a pump feeder tube 310. The pump feeder tube 310 can be coupled with the pump 308 using a fastener 314, such as a clip. The pump 308 includes a fan 316 which is operable to be rotated to promote flow of the sample gas. In at least one example, the sample gas can then be received in a restrictor feed tube 318, passed through a restrictor 320 which is received in a restrictor housing 322, and passed through a restrictor return tube 324. The restrictor 320 can be operable to restrict gas flow by creating a pressure differential. In at least some examples, the restrictor 320 can be incorporated into the calibration manifold 356. In other examples, the gas analyzer unit 300 may not include the restrictor feed tube, restrictor, restrictor housing, or restrictor return tube. In this example, the calibration manifold 356 can incorporate the function of the restrictor 320 by including a restrictor aperture to restrict sample gas flow to create the pressure differential.

The restrictor 320 and/or calibration manifold 356 can be utilized to control the speed and/or quantity of the sample gas received by the gas sensor module 100. The sample gas can then pass through the pump 308 and out the pump delivery tube 312.

The gas sensor assembly 10 can include a sample tube 352 fluidly connected to the gas delivery device 50 and the gas sensor module 100 operable to receive the sample gas. For example, the sample tube 352 can be fluidly connected to the pump delivery tube 312. In at least one example, at least a portion of the sample tube 352 can be a water-permeable tube made of a sulfonated tetrafluoroethylene based fluoropolymer-copolymer (e.g. a Nafion™ tube). As illustrated in FIG. 1A, the gas sensor assembly 10 can additionally include a humidity component 354 and a calibration manifold 356. The humidity component 354, the water-permeable tube portion of the sample tube 352, the calibration manifold 356, any other suitable components for example to control the temperature and/or pressure, or any combination thereof can control at least one of temperature, relative humidity, and pressure, facilitating the generation of calibration functions that not only provide a more accurate measurement of gas, such as NO, in specific sub-ranges but also enable compensation for different temperatures, pressures, and/or relative humidity values. For example, the humidity component 354, the water-permeable tube portion of the sample tube 352, and/or the calibration manifold 356 can lower the humidity of the gas sample to increase the calibration stability of the gas sensor module 100. A gas analyzer subframe 357 can be included to house at least a portion of the humidity component 354, the water-permeable tube portion of the sample tube 352, and/or the calibration manifold 356. One or more fasteners 358 can retain at least one of the humidity component 354, the water-permeable tube portion of the sample tube 352, and/or the calibration manifold 356 within the gas analyzer subframe 357. The fasteners 358 can be, for example, screws, adhesives, and/or nuts and bolts.

The gas sensor assembly 10 additionally can include a high differential link tube 360 and a low differential link tube 362. In at least one example, the gas sensor assembly 10 can include an ambient air pressure link tube 364 which is fluidly connected with external atmosphere or ambient air. To provide ambient air, the gas sensor assembly 10 can include an ambient air inlet tube 368 which is fluidly connected with exterior of the gas sensor assembly 10 to provide ambient air. A filter 372 is coupled with an end of the ambient air inlet tube 368 opposite the end connected with the exterior of the gas sensor assembly 10. The filter 372 can filter the ambient air to prevent particles or other substances which may affect the gas sensor module 100 from determining accurate measurements of the sample gas. A connector tube 366 can be included to fluidly connect the water-permeable tube portion of the sample tube 352 with the calibration manifold 356. Additionally, in at least one example, a filter tube 370 can be fluidly connected with the filter 372 to provide a passage of the ambient air to the water-permeable tube portion of the sample tube 352.

The sample gas is received through the port 206 of the assembly inner housing 200. The port 206 is fluidly connected with the inlet 119 of the gas sensor module 100, and the sample gas is received within the sample chamber 101 of the gas sensor module 100.

Also provided herein is a method for providing a gas sensor module for use in a therapeutic gas delivery device. In some examples, the method may include calibrating the plurality of sensors in the gas sensor module, and providing electrical potential across the plurality of sensors to maintain the calibration of the plurality of sensors. The calibration of the plurality of sensors can be maintained for at least 1 month, at least 3 months, at least 6 months, or at least 1 year. The electrical potential may be provided by an apparatus with a voltage source, such as a battery. In some examples, the method may further include removing the apparatus/voltage source prior to or simultaneously with the installation of the gas sensor module in the therapeutic gas delivery device. The gas sensor module can be installed within the assembly inner housing and assembly outer housing in the therapeutic gas delivery device. In some examples, the installation of the gas sensor module results in less than 5 minutes of down time in the measurement of at least one property of a sample gas from the therapeutic gas delivery device. In other examples, the installation of the gas sensor module results in no down time in the delivery of therapeutic gas to a patient.

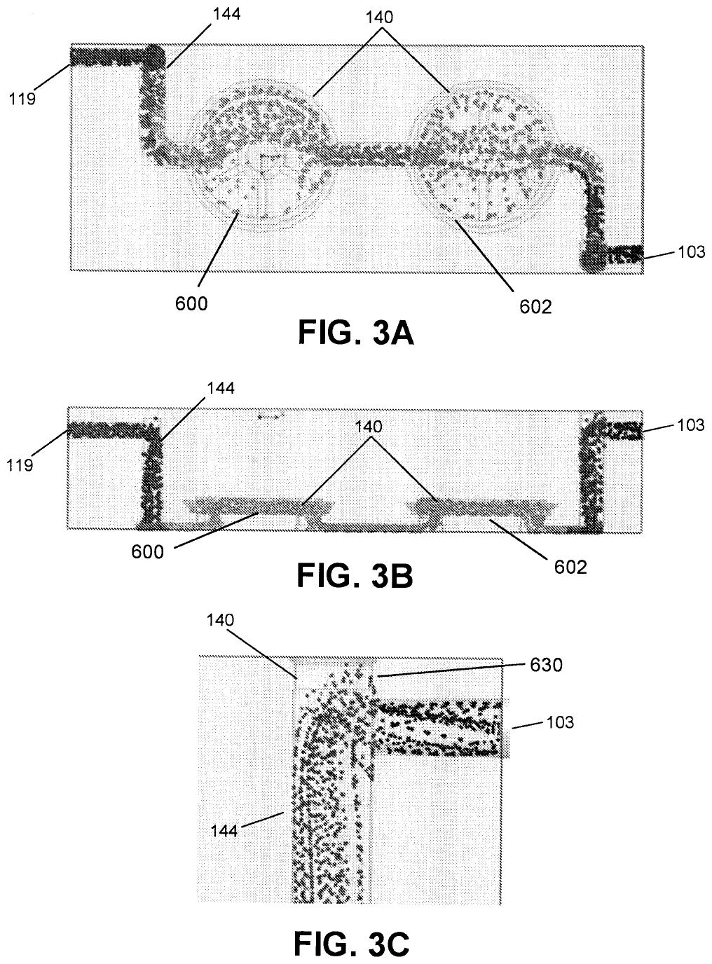

FIGS. 3A-3C show models of gas flow through an exemplary gas sensor module, wherein the color gradient represents the velocity of the gas through the flow passage and the sensor chambers. The sensor chambers 140 can include a first sensor chamber 600, a second sensor chamber 602, and a third sensor chamber 630. As can be seen in FIGS. 3A-3B, the gas flow velocity is highest at the inlet 119 to the sample chamber. When the gas enters the first sensor chamber 600, the velocity of the gas slows. The velocity continues to decrease as the gas flows through the second sensor chamber 602 and is slowest at the outlet 103. FIG. 3C shows that the gas may flow upward above the outlet into a third sensor chamber 630 before exiting the sample chamber through the outlet 103.

FIGS. 4A-4D show models of gas flow through another exemplary gas sensor module. Here, as in FIGS. 3A-3C, the velocity of the gas is highest in the flow passage 144 between the inlet 119 and the first sensor 122. The velocity of the gas is lowest within the sensor chambers 140 and at the outlet 103. As can be seen in FIG. 4D, the gas may flow upward above the outlet 103 into a third sensor chamber, for example, a temperature/humidity sensor chamber.

As illustrated in FIGS. 4A-4D, the flow passage 144 can include multiple turns and straight pathways before, between, and after the first sensor chamber 600, the second sensor chamber 602, and the third sensor chamber 630. The turns and straight pathways can increase and/or decrease the velocity of the sample gas (e.g., sample gas particles) thereby allowing homogenous mixing (e.g., the concentration of breathing gas and therapeutic gas is mixed such that the concentration profiles are substantially constant throughout the sensor chambers 140). For example, the flow passage 144 allows the sample gas to mix such that there are not pockets (e.g., spots within the flow passage 144) in the flow passage 144 where the concentration of the sample gas substantially deviates from the concentration of the sample gas in other areas of the flow passage 144. The turns and straight paths can allow for turbulent flow in the flow passage 144, thereby allowing for homogenous mixing of the sample gas. The turbulent flow can provide sufficient mixing of the sample gas such that the sample gas tested in the sensors is mixed at substantially the same ratio as the gas mixture delivered to the patient. The turbulent flow causes sample gas particles to move in different directions and at different velocities, thereby influencing homogenous mixing of the sample gas. It is further contemplated that the flow through the flow passage 144 may not reach turbulent flow but can still provide sufficient mixing such that the sample gas is a homogenous mixture.

In some examples, the flow passage 144 ensures that the sample gas is homogenously mixed when entering the first sensor chamber 600 (e.g., if the same concentrations of breathing gas and therapeutic gas are entering the inlet 119, the concentration of the sample gas entering the first sensor chamber 600 will be substantially constant). In some examples, the flow passage 144 ensures that the sample gas is homogenously mixed when entering the second sensor chamber 602 (e.g., the therapeutic gas and breathing gas exiting the first sensor chamber 600 is mixed by the flow passage 144 between the first sensor chamber 600 and second sensor chamber 602 such that the concentration of the sample gas entering the second sensor chamber 602 is substantially constant). In some examples, the flow passage 144 ensures that the sample gas is homogenously mixed when entering the third sensor chamber 630 (e.g., the therapeutic gas and breathing gas exiting the second sensor chamber 602 is mixed by the flow passage 144 between the second sensor chamber 602 and the third sensor chamber 630 such that the concentration of the sample gas entering the third sensor chamber 630 is substantially constant).

The sample gas can enter the sample chamber 101 at the inlet 119. The sample gas can have a velocity of about 0.3 meters/second (m/s) to about 1 m/s at the inlet 119. In some examples, the sample gas can have a velocity of about 0.3 m/s to about 0.4 m/s, about 0.4 m/s to about 0.5 m/s, about 0.5 m/s to about 0.6 m/s, about 0.6 m/s to about 0.7 m/s, about 0.7 m/s to about 0.8 m/s, about 0.8 m/s to about 0.9 m/s, or about 0.9 m/s to about 1 m/s when the sample gas enters the inlet 119. The flow passage 144 of the sample chamber 101 can be configured to allow for mixing of the sample gas and thereby allow a homogenous mixture of the sample gas to be tested in the first sensor chamber 600, second sensor chamber 602, and third sensor chamber 630.

Once the sample gas enters the inlet 119, the sample gas can flow downward along a 90 degree turn 400. The 90 degree turn 400 can slow the velocity of the sample gas to a velocity of about 0 m/s to about 0.3 m/s. The 90 degree turn 400 allows for further mixing of the sample gas thereby forming a homogenous mixture of the breathing gas and therapeutic gas. The sample gas can then flow along a straight vertical path 402, as illustrated in FIGS. 4A-4D. The velocity of the sample gas can increase along the straight vertical path 402. In some examples, the velocity of the sample gas along the straight vertical path 402 can be about 0.1 m/s to about 0.2 m/s, about 0.2 m/s to about 0.3 m/s, about 0.3 m/s to about 0.4 m/s, about 0.4 m/s to about 0.5 m/s, about 0.5 m/s to about 0.6 m/s, about 0.6 m/s to about 0.7 m/s, about 0.7 m/s to about 0.8 m/s, or any velocity or range of velocities therebetween.

The sample gas can then flow to a 90 degree turn 404 into a straight horizontal path 406, as illustrated in FIGS. 4A-4D. The 90 degree turn 404 can slow the velocity of the sample gas. For example, the 90 degree turn 404 can slow the velocity of the sample gas to about 0 m/s to about 0.3 m/s. The sample gas can then flow along the straight horizontal path 406. The velocity of the sample gas can increase along the straight horizontal path 406. In some examples, the velocity of the sample gas along the straight horizontal path 406 can be about 0.3 m/s to about 0.4 m/s, about 0.4 m/s to about 0.5 m/s, about 0.5 m/s to about 0.6 m/s, about 0.6 m/s to about 0.7 m/s, about 0.7 m/s to about 0.8 m/s, about 0.8 m/s to about 0.9 m/s, about 0.9 m/s to about 1 m/s, about 1 m/s to about 1.1 m/s, about 1.1 m/s to about 1.2 m/s, or any velocity or range of velocities therebetween.

The sample gas can then enter a vertical 90 degree turn 408. As illustrated in FIGS. 4A-4C, the vertical 90 degree turn 408 can be operable to slow the velocity of the sample gas. For example, the velocity of the sample gas when exiting the vertical 90 degree turn 408 can be about 0 m/s to about 0.1 m/s, about 0.1 m/s to about 0.2 m/s, or any velocity or range of velocities therebetween. After the vertical 90 degree turn 408, the sample gas can flow along a short straight vertical path 410. The short straight vertical path 410 can be short enough such that the sample gas does not gain significant velocity along the short straight vertical path 410. In this manner, the homogenous mixture of the sample gas can be ensured when tested at the first sensor chamber 600. In some examples, the short straight vertical path 410 can have a length that is about one, two, three, four, five, six, seven, eight, nine, ten, or more times less than the length of the other straight paths in the flow passage 144 (e.g., straight vertical path 402 and/or straight horizontal path 406).

The sample gas can then flow from the short straight vertical path 410 to another 90 degree turn 412. The 90 degree turn 412 can be a horizontal turn (e.g., receives sample gas traveling in a vertical direction and redirects the sample gas to travel in a horizontal direction). The 90 degree turn 412 can be operable to provide the sample gas to the first sensor chamber 600 such that the sample gas can be detected and/or tested by the sensors 118, 122. The 90 degree turn 412 can further slow the velocity of the sample gas. For example, the sample gas exiting the 90 degree turn 412 can be about 0 m/s to about 0.05 m/s, about 0.05 m/s to about 0.1 m/s, about 0.1 m/s to about 0.15 m/s, about 0.15 m/s to about 0.2 m/s, or any velocity or range of velocities therebetween.

The sample gas can then flow through the first sensor chamber 600 and be detected and/or tested by the one or more sensors 118, 122 in the first sensor chamber 600. The sample gas can flow through first sensor chamber 600 along straight horizontal path 414. The velocity of the sample gas can remain low along the straight horizontal path such that the sample gas diffuses throughout the one or more sensors 118, 122. For example, the velocity of the sample gas along the straight horizontal path 414 can be about 0 m/s to about 0.05 m/s, about 0.05 m/s to about 0.1 m/s, about 0.1 m/s to about 0.15 m/s, about 0.15 m/s to about 0.2 m/s, or any velocity or range of velocities therebetween. After being detected and/or tested by the sensors 118, 122 in the first sensor chamber 600, the sample gas can exit the first sensor chamber 600 at 90 degree turn 416. 90 degree turn 416 can be a vertical turn, as illustrated, for example, in FIG. 4C. The sample gas can increase in velocity after exiting the 90 degree turn 416 and traveling along a vertical path. For example, the velocity of the sample gas can increase to about 0.3 m/s to about 0.4 m/s, about 0.4 m/s to about 0.5 m/s, about 0.5 m/s to about 0.6 m/s, about 0.6 m/s to about 0.7 m/s, about 0.7 m/s to about 0.8 m/s, about 0.8 m/s to about 0.9 m/s, about 0.9 m/s to about 1.0 m/s, or any velocity or range of velocities therebetween. In some examples, the sample gas can then pass through 90 degree turn 418. 90 degree turn 418 can be a 90 degree turn or can be a turn having an angle less than 90 degrees. In some examples, the velocity of the sample gas increases further at 90 degree turn 418. 90 degree turn 418 can change the flow direction of the sample gas from a vertical flow path to a horizontal flow path along straight horizontal path 420. In some examples, the velocity of the sample gas exiting 90 degree turn 418 can be about 0.4 m/s to about 0.5 m/s, about 0.5 m/s to about 0.6 m/s, about 0.6 m/s to about 0.7 m/s, about 0.7 m/s to about 0.8 m/s, about 0.8 m/s to about 0.9 m/s, about 0.9 m/s to about 1.0 m/s, or any velocity or range of velocities therebetween.

The sample gas can then flow along straight horizontal path 420. In some examples, the sample gas can have a velocity of about 0.4 m/s to about 0.5 m/s, about 0.5 m/s to about 0.6 m/s, about 0.6 m/s to about 0.7 m/s, about 0.7 m/s to about 0.8 m/s, about 0.8 m/s to about 0.9 m/s, about 0.9 m/s to about 1.0 m/s, about 1.0 m/s to about 1.1 m/s, about 1.1 m/s to about 1.2 m/s, about 1.2 m/s to about 1.3 m/s, about 1.3 m/s to about 1.4 m/s, or any velocity or range of velocities therebetween. In some examples, increasing the velocity of the sample gas along the flow passage 144 between 90 degree turn 416 and 90 degree turn 422 further promotes turbulent flow and mixing of the sample gas.

The sample gas can then flow to 90 degree turn 422. At 90 degree turn 422, the sample gas can be directed to flow in a vertical direction. 90 degree turn 422 can slow the velocity of the sample gas. For example, the velocity of the sample gas exiting 90 degree turn 422 can be about 0 m/s to about 0.05 m/s, about 0.05 m/s to about 0.1 m/s, about 0.1 m/s to about 0.15 m/s, about 0.15 m/s to about 0.2 m/s, or any velocity or range of velocities therebetween. The sample gas can then flow through a short vertical path 423. The length of the short vertical path 423 can be less than the length of other straight flow paths in the flow passage 144. For example, the short vertical path 423 can have a length about one, two, three, four, five, six, seven, eight, nine, ten, or more times shorter than the length of the other straight paths (e.g., straight vertical path 402, straight horizontal path 406, and straight horizontal path 420). The short vertical path 423 can be short enough such that the velocity of the sample gas does not increase significantly. In this manner, homogenously mixed sample gas is delivered to the second sensor chamber 602.

The sample gas can then enter 90 degree turn 424. 90 degree turn can redirect the gas flow from a vertical flow direction along short vertical path 423 to a horizontal direction along straight horizontal path 426. The sample gas exiting 90 degree turn 424 enters into the second sensor chamber 602. For example, the 90 degree turn 424 is the inlet to the second sensor chamber 602. The gas can be detected and/or tested in the second sensor chamber 602 by the one or more sensors 118, 122. As illustrated in FIG. 4C, the sample gas exiting the 90 degree turn 424 can have a low velocity (e.g., 90 degree turn 424 can slow the velocity of the sample gas). For example, the sample gas exiting the 90 degree turn 424 can have a velocity of about 0 m/s to about 0.05 m/s, about 0.05 m/s to about 0.1 m/s, about 0.1 m/s to about 0.15 m/s, about 0.15 m/s to about 0.2 m/s, or any velocity or range of velocities therebetween. The gas can then flow through the second sensor chamber 602 along straight horizontal path 426. The velocity of the sample gas along straight horizontal path 426 can be about 0 m/s to about 0.05 m/s, about 0.05 m/s to about 0.1 m/s, about 0.1 m/s to about 0.15 m/s, about 0.15 m/s to about 0.2 m/s, or any velocity or range of velocities therebetween. In some examples, the sample gas flowing along the straight horizontal path 426 maintains a low velocity such that the sample gas diffuses along the one or more sensors 118, 122.

The sample gas can exit the second sensor chamber 602 at 90 degree turn 428. 90 degree turn 428 can redirect the sample gas from a horizontal flow direction along straight horizontal path 426 to a vertical flow direction (e.g., along a short vertical path between 90 degree turn 428 and 90 degree turn 430). In some examples, the velocity of the sample gas can increase as the sample gas exits 90 degree turn 428. For example, the velocity of the sample gas can increase as it exits 90 degree turn 428 and enters a short vertical path between 90 degree turn 428 and 90 degree turn 430. In some examples, the velocity of the sample gas at the exit of 90 degree turn 428 and into the short vertical path between 90 degree turn 428 and 90 degree turn 430 is about 0.3 m/s to about 0.4 m/s, about 0.4 m/s to about 0.5 m/s, about 0.5 m/s to about 0.6 m/s, about 0.6 m/s to about 0.7 m/s, about 0.7 m/s to about 0.8 m/s, about 0.8 m/s to about 0.9 m/s, about 0.9 m/s to about 1.0 m/s, or any velocity or range of velocities therebetween.

The flow passage 144 can then turn from a vertical flow path (e.g., between 90 degree turn 428 and 90 degree turn 430) to a horizontal path. 90 degree turn 430 can change the direction of the flow of the sample gas from a vertical direction to a horizontal direction along straight horizontal path 432. In some examples, the velocity of the sample gas can continue increasing along straight horizontal path 432. In some examples, the velocity of the sample gas along straight horizontal path 432 can be about 0.4 m/s to about 0.5 m/s, about 0.5 m/s to about 0.6 m/s, about 0.6 m/s to about 0.7 m/s, about 0.7 m/s to about 0.8 m/s, about 0.8 m/s to about 0.9 m/s, about 0.9 m/s to about 1 m/s, about 1 m/s to about 1.1 m/s, about 1.1 m/s to about 1.2 m/s, about 1.2 m/s to about 1.3 m/s, about 1.3 m/s to about 1.4 m/s, or any velocity or range of velocities therebetween. In some examples, the velocity is increased such that the sample gas moves through the sample chamber 101 more quickly, thereby allowing additional samples of sample gas to enter the sample chamber 101 and provide continued monitoring of the properties (e.g., parameters) of the sample gas.

The sample gas can then be redirected along a straight vertical path 436 by 90 degree turn 434. The velocity of the sample gas can be decreased at 90 degree turn 434. In some examples, the velocity of the sample gas can be decreased such that the velocity of the sample gas exiting 90 degree turn 434 is about 0 m/s to about 0.05 m/s, about 0.05 m/s to about 0.1 m/s, about 0.1 m/s to about 0.15 m/s, about 0.15 m/s to about 0.2 m/s, about 0.2 m/s to about 0.25 m/s, about 0.25 m/s to about 0.3 m/s, about 0.3 m/s to about 0.35 m/s, about 0.35 m/s to about 0.4 m/s, or any velocity or range of velocities therebetween. The sample gas can then flow through straight vertical path 436 to 90 degree turn 438. In some examples, 90 degree turn 438 can be part of a T-path (e.g., part of the sample gas can flow through outlet 103 and part of the sample gas can flow to a third sensor chamber 630). In some examples, the sample gas can flow up to a third sensor chamber 630 where the sample gas is tested and/or detected by a sensor 118 before flow through outlet 103. In some examples, 90 degree turn 438 can slow the velocity of the sample gas. For example, the velocity of the sample gas at 90 degree turn 438 (or T-path) can be about 0 m/s to about 0.05 m/s, about 0.05 m/s to about 0.1 m/s, about 0.1 m/s to about 0.15 m/s, about 0.15 m/s to about 0.2 m/s, or any velocity or range of velocities therebetween. In some examples, the sensor 118 in the third sensor chamber 630 can be a humidity sensor and/or temperature sensor.

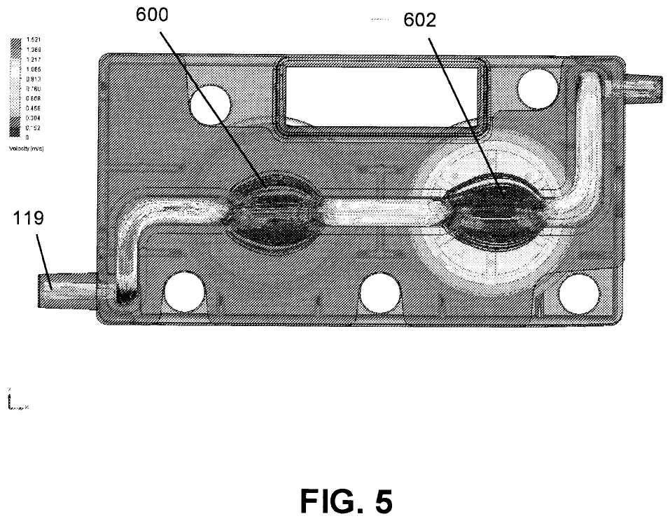

FIG. 5 shows a model of the gas flow through another exemplary gas sensor module. FIG. 5 shows the pathways of gas particles and their velocities through the gas sensor module. As can be seen in FIG. 5, the velocity of the gas is highest at the inlet and is lowest within the sensor chambers. For example, the velocity of the sample gas can be about 0.4 m/s to about 1.6 m/s from the inlet 119 to the first sensor chamber 600. The velocity of the sample gas can be about 0 m/s to about 0.3 m/s in the first sensor chamber 600. The velocity of the sample gas can be about 0.3 m/s to about 1.1 m/s between the first sensor chamber 600 and the second sensor chamber 602. The velocity of the sample gas in the second sensor chamber 602 can be about 0 m/s to about 0.3 m/s. The velocity of the sample gas between the second sensor chamber 602 and the outlet 103 can be about 0.3 m/s to about 1.1 m/s.

It will be appreciated that the velocities described herein with respect to FIGS. 4A-4C and 5 are exemplary only. The velocities may be different in different situations due to different gas mixtures, different pump speeds, different pressures, or other differing parameters. However, the gas sensor module described herein can provide significant benefits regardless of the type of gas mixture, pump speed, pressure, other parameters due to the flow passage 144 which is operable to provide turbulent flow and homogenous mixture of the sample gas, thereby allowing for a homogenous mixture of the sample gas to be accurately tested. In this manner, the sample gas tested is substantially the sample as the gas mixture delivered to the patient, thereby allowing for accurate determination of the properties of the gas mixture being delivered to the patient.

FIG. 6A-6B show the flow passage 144 through an exemplary gas sensor module. The flow passage 144 begins at the inlet 119, followed by a first 90 degree turn 604 along the vertical axis, followed by a second 90 degree turn 606 along the horizontal axis, a third 90 degree turn 608 along the horizontal axis, a fourth 90 degree turn 610 along the vertical axis, a fifth 90 degree turn 611 along the horizontal axis, a sixth 90 degree turn 612 along the vertical axis, a seventh 90 degree turn 614 along the horizontal axis, an eighth 90 degree turn 616 along the vertical axis, a nineth 90 degree turn 618 along the horizontal axis, a tenth 90 degree turn 620 along the vertical axis, an eleventh 90 degree turn 622 along the horizontal axis, a twelfth 90 degree turn 624 along the horizontal axis, a thirteenth 90 degree turn 626 along the vertical axis, and a fourteenth 90 degree turn 628 along the horizontal axis, followed by the outlet 103. In some examples, 90 degree turn 628 can be part of a T-path 632, as illustrated in FIG. 6A. The T-path 632 can be operable to provide all or a portion of the sample gas to the third sensor chamber 630 described herein, such that a sensor in the third sensor chamber can detect and/or test the sample gas before the sample gas exits the outlet 103.

As described herein, a vertical turn is defined as a turn that changes the direction of the flow either to or from a vertical direction. For example, a vertical turn means that the flow passage 144 turns either up or down along the vertical axis or turns from a vertical direction to a horizontal direction along the horizontal axis. A vertical turn can change the flow path of the flow passage 144 from a vertical flow path to a horizontal flow path and/or from a horizontal flow path to a vertical flow path. A vertical turn can change the direction of the flow by changing the horizontal plane of the flow direction (e.g., a vertical turn changes the horizontal plane that the flow is in from the inlet of the vertical turn to the outlet of the vertical turn). For example, both 90 degree turn 604 and 90 degree turn 606 are defined as vertical turns as described herein. 90 degree turn 604 is a vertical turn that changes the direction of the flow passage 144 along the vertical axis. 90 degree turn 606 is a vertical turn that changes the direction of the flow passage 144 along the horizontal axis. A horizontal turn is defined as a turn that changes the direction of the flow passage 144 within the same horizontal plane. A horizontal turn, as defined herein, only changes the direction of the flow passage along the horizontal axis. For example, a horizontal turn does not change the vertical direction or path of the flow in any way. For example, 90 degree turn 608 and 90 degree turn 624 are horizontal turns as defined herein. It will be appreciated that turns can be both horizontal and vertical turns (e.g., can change both the vertical direction flow path and the horizontal direction flow path). For example, a turn that is both a horizontal and a vertical turn can change the flow path in the vertical direction (e.g., along a vertical axis) and the flow path in a horizontal direction (e.g., along the horizontal axis).

In some examples, the sixth 90 degree turn 611 and seventh 90 degree turn 612 are not turns of a tubular flow passage but rather are caused by an upper ceiling or cover of the first sensor chamber 600. In some examples, the nineth 90 degree turn 618 and the tenth 90 degree turn 620 are not turns of a tubular flow passage but rather are caused by an upper ceiling or cover of the second sensor chamber 602. For example, when the sample gas enters the first sensor chamber 600 and second sensor chamber 602, the sample gas can be free to move through the first sensor chamber 600 and second sensor chamber 602 without be constrained by a tubular flow passage, thereby allowing even diffusion across the sensors 118, 122 in the first sensor chamber 600 and the second sensor chamber 602. The sample gas can be pushed through the first sensor chamber 600 and the second sensor chamber 602 by the pressure supplied to the sample gas in the flow passage 144. For example, 90 degree turn 610 can form the inlet to the first sensor chamber 600 and 90 degree turn 614 can form the outlet of the first sensor chamber 600. 90 degree turn 616 can form the inlet of the second sensor chamber 602 and 90 degree turn 622 can form the outlet of the second sensor chamber 602. In some examples, an upper exit portion of the T-path 632 can form the inlet and the outlet to the third sensor chamber 630.

In some examples, when fifth 90 degree turn 611, sixth 90 degree turn 612, nineth 90 degree turn 618, and tenth 90 degree turn 620 are not part of the tubular flow path but rather are caused by a ceiling or cover of the first sensor chamber 600 and the second sensor chamber 602, the flow path of the flow passage can be as follows: sample gas can enter the inlet 119 in a horizontal direction, first 90 degree turn 604 can direct the sample gas vertically downward, second 90 degree turn 606 can direct the sample gas from the vertical direction to a horizontal direction, third 90 degree turn 608 can change the direction of the flow of the sample gas within the horizontal plane along the horizontal axis, fourth 90 degree turn 610 can direct the sample gas from a horizontal direction to a vertical direction and into the first sensor chamber 600, seventh 90 degree turn 614 can receive the sample gas from the sensor chamber in a vertical direction and direct the sample gas in a horizontal direction, eighth 90 degree turn 616 can receive the sample gas in a horizontal direction and direct the gas into the second sensor chamber 602 in a vertical direction, eleventh 90 degree turn 622 can receive the sample gas from the second sensor chamber in a vertical direction and direct the gas in a horizontal direction, twelfth 90 degree turn 624 can receive the sample gas in a horizontal direction and change the horizontal flow direction of the sample gas, thirteenth 90 degree turn 626 can receive the sample gas in a horizontal direction and direct the sample gas in a vertical direction, fourteenth 90 degree turn 628 can receive the sample gas in a vertical direction and direct the sample gas in a horizontal direction to the outlet 103.

It will be appreciated that while 90 degree turns in the flow passage 144 are described herein other types of turns can be used to increase and/or decrease the velocity of the sample gas particles to provide a homogenous mixture of sample gas to the sensor chambers 140. For example, the turns can have angles of about 5 degrees to about 10 degrees, about 10 degrees to about 15 degrees, about 15 degrees to about 20 degrees, about 20 degrees to about 25 degrees, about 25 degrees to about 30 degrees, about 30 degrees to about 35 degrees, about 35 degrees to about 40 degrees, about 40 degrees to about 45 degrees, about 45 degrees to about 50 degrees, about 50 degrees to about 55 degrees, about 55 degrees to about 60 degrees, about 60 degrees to about 65 degrees, about 65 degrees to about 70 degrees, about 70 degrees to about 75 degrees, about 75 degrees to about 80 degrees, about 80 degrees to about 85 degrees, about 85 degrees to about 90 degrees, about 90 degrees to about 95 degrees, about 95 degrees to about 100 degrees, about 100 degrees to about 105 degrees, about 105 degrees to about 110 degrees, about 110 degrees to about 115 degrees, about 115 degrees to about 120 degrees, about 120 degrees to about 125 degrees, about 125 degrees to about 130 degrees, or more. In some examples, the angle of the turns in the flow passage 144 can be configured to increase and/or decrease the velocity of the sample gas such that the sensors 118, 122 can detect and/or test a homogenous gas mixture.

It will be appreciated that while certain numbers of turns are shown in the flow passage 144, different numbers of turns can be used to increase and/or decrease the velocity of sample gas, thereby providing a homogenous sample gas mixture to the sensor chambers 140. In some examples, the flow passage 144 can have more or less turns than the amount of turns described herein.

In some examples, the flow passage 144 can have a diameter of about less than 0.5 inches. In some examples, the flow passage 144 can have a diameter of about 0.01 inches to about 0.02 inches, about 0.02 inches to about 0.03 inches, about 0.03 inches to about 0.04 inches, about 0.04 inches to about 0.05 inches, about 0.05 inches to about 0.06 inches, about 0.06 inches to about 0.07 inches, about 0.07 inches to about 0.08 inches, about 0.08 inches to about 0.09 inches, about 0.09 inches to about 0.1 inches, about 0.1 inches to about 0.11 inches, about 0.11 inches to about 0.12 inches, about 0.12 inches to about 0.13 inches, about 0.13 inches to about 0.14 inches, about 0.14 inches to about 0.15 inches, about 0.15 inches to about 0.16 inches, about 0.16 inches to about 0.17 inches, about 0.17 inches to about 0.18 inches, about 0.18 inches to about 0.19 inches, about 0.19 inches to about 0.2 inches, about 0.2 inches to about 0.25 inches, about 0.25 inches to about 0.3 inches, about 0.3 inches to about 0.35 inches, about 0.35 inches to about 0.4 inches, about 0.4 inches to about 0.45 inches, about 0.45 inches to about 0.5 inches, or more. In some examples, the flow passage 144 can have a constant diameter from the inlet 119 to the first sensor chamber 600. In some examples, the flow passage 144 can have a constant diameter from the first sensor chamber 600 to the second sensor chamber 602. In some examples, the flow passage 144 can have a constant diameter from the second sensor chamber 602 to the third sensor chamber 630. In some examples, the flow passage 144 can have a constant diameter from the third sensor chamber 630 to the outlet 103. In some examples, the diameter of the flow passage 144 can vary throughout the flow passage 144. For example, the diameter can increase or decrease along the straight pathways and/or through the turns. In some examples, the flow passage 144 can have an increasing diameter along portions of the flow passage 144 where velocity of the sample gas is to be decreased. In some examples, the flow passage can have a decreasing diameter along portions of the flow passage 144 where velocity of the sample gas is to be increased.

In some examples, the flow passage 144 can have a total length (e.g., length from the inlet 119 to the outlet 103 including the portions of the flow passage 144 passing through the sensor chambers 140) of about less than 5 inches. In some examples, the flow passage 144 can have a total length of about less than 0.5 inches, about 0.5 inches to about 0.6 inches, about 0.6 inches to about 0.7 inches, about 0.7 inches to about 0.8 inches, about 0.8 inches to about 0.9 inches, about 0.9 inches to about 1 inch, about 1 inch to about 1.1 inches, about 1.1 inches to about 1.2 inches, about 1.2 inches to about 1.3 inches, about 1.3 inches to about 1.4 inches, about 1.4 inches to about 1.5 inches, about 1.5 inches to about 1.6 inches, about 1.6 inches to about 1.7 inches, about 1.7 inches to about 1.8 inches, about 1.8 inches to about 1.9 inches, about 1.9 inches to about 2.0 inches, about 2.0 inches to about 2.1 inches, about 2.1 inches to about 2.2 inches, about 2.2 inches to about 2.3 inches, about 2.3 inches to about 2.4 inches, about 2.4 inches to about 2.5 inches, about 2.5 inches to about 2.6 inches, about 2.6 inches to about 2.7 inches, about 2.7 inches to about 2.8 inches, about 2.8 inches to about 2.9 inches, about 2.9 inches to about 3.0 inches, about 3.0 inches to about 3.1 inches, about 3.1 inches to about 3.2 inches, about 3.2 inches to about 3.3 inches, about 3.3 inches to about 3.4 inches, about 3.4 inches to about 3.5 inches, about 3.5 inches to about 3.6 inches, about 3.6 inches to about 3.7 inches, about 3.7 inches to about 3.8 inches, about 3.8 inches to about 3.9 inches, about 3.9 inches to about 4.0 inches, about 4.0 inches to about 4.1 inches, about 4.1 inches, about 4.1 inches to about 4.2 inches, about 4.2 inches to about 4.3 inches, about 4.3 inches to about 4.4 inches, about 4.4 inches, about to about 4.5 inches, about 4.5 inches to about 4.6 inches, about 4.6 inches to about 4.7 inches, about 4.7 inches to about 4.8 inches, about 4.8 inches to about 4.9 inches, about 4.9 inches to about 5.0 inches, or more.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

What is claimed is:

1. A gas sensor module for a therapeutic gas delivery device, the gas sensor module comprising:
a sample inlet operable to receive a sample gas comprising a mixture of a breathing gas and a therapeutic gas from a sample line connected to an inspiratory line of the therapeutic gas delivery device; and
a sample chamber forming a flow passage fluidly connected with the sample inlet and operable to receive the sample gas, the sample chamber includes a plurality of sensor chambers operable to be in communication with one or more corresponding sensors operable to measure at least one property of the sample gas,
wherein the flow passage is operable to direct a flow of the sample gas from the sample inlet to the plurality of sensor chambers, between each of the plurality of sensor chambers, and from the plurality of sensor chambers to an outlet, and
wherein the flow passage comprises one or more turns prior to each of the plurality of sensor chambers, each turn having an angle operable to promote mixing of the sample gas and promote diffusion of the sample gas onto each of the one or more corresponding sensors.

2. The gas sensor module of claim 1, wherein the mixing of the sample gas is such that a concentration of the breathing gas and the therapeutic gas in the sample gas flowing over the plurality of sensor chambers is substantially the same as the concentration of the breathing gas and the therapeutic gas in the inspiratory line.

3. The gas sensor module of claim 1, wherein the flow passage reduces or prevents rotating eddies and dead zones in the sample gas flow through the sample chamber.

4. The gas sensor module of claim 1, wherein the flow passage creates a low back pressure or resistance to flow through the sample chamber.

5. The gas sensor module of claim 1, wherein the mixing of the sample gas provides a turbulent delivery of the sample gas to a surface of each of the one or more corresponding sensors.

6. The gas sensor module of claim 1, wherein a volume of sample gas in the sample chamber is less than a volume of sample gas that would be needed in a sample chamber without mixing.

7. The gas sensor module of claim 1, wherein the angle of the one or more turns is about 90 degrees.

8. The gas sensor module of claim 1, wherein the one or more turns are operable to change direction of the flow passage within the sample chamber.

9. The gas sensor module of claim 1, wherein the sample chamber comprises an inner housing having an upper portion, a lower portion, a vertical axis, and a horizontal axis transverse to the vertical axis, wherein at least one of the one or more turns is along the vertical axis and/or the horizontal axis.

10. The gas sensor module of claim 9, wherein the one or more turns are about 90 degrees along the vertical axis and/or the horizontal axis of the sample chamber.

11. The gas sensor module of claim 9, wherein at least one of the plurality of sensor chambers is located in the lower portion of the sample chamber and the sample inlet is located in the upper portion of the sample chamber.

12. The gas sensor module of claim 11, wherein the plurality of sensor chambers includes a first sensor chamber comprising a first sensor in the lower portion of the sample chamber, and wherein the flow passage includes about one to about four turns, each having angles of about 90 degrees, between the sample inlet in the upper portion of the sample chamber and an inlet of the first sensor chamber.

13. The gas sensor module of claim 12, wherein the flow passage between the sample inlet and the first sensor chamber includes a first vertical turn along the vertical axis, a second vertical turn along the horizontal axis, a first horizontal turn along the horizontal axis subsequent to the second vertical turn, and a third vertical turn along the vertical axis.

14. The gas sensor module of claim 12, wherein the first sensor chamber is a nitrogen dioxide sensor chamber and the first sensor is a nitrogen dioxide sensor.

15. The gas sensor module of claim 12, wherein the plurality of sensor chambers includes a second sensor chamber comprising a second sensor in the lower portion of the sample chamber, and wherein the flow passage includes about one to about four turns, each having angles of about 90 degrees, between an outlet of the first sensor chamber and an inlet of the second sensor chamber.

16. The gas sensor module of claim 15, wherein the flow passage between the outlet of the first sensor chamber and the inlet of the second sensor chamber includes a fourth vertical turn along the horizontal axis and a fifth vertical turn along the vertical axis subsequent to the fourth vertical turn.

17. The gas sensor module of claim 15, wherein the second sensor chamber is a nitric oxide sensor chamber and the second sensor is a nitric oxide sensor.

18. The gas sensor module of claim 15, wherein the plurality of sensor chambers includes a third sensor chamber comprising a third sensor in the upper portion of the sample chamber, and wherein the flow passage includes about one to about four turns, each having angles of about 90 degrees, between an outlet of the second sensor chamber and an opening of the third sensor chamber.

19. The gas sensor module of claim 18, wherein the flow passage between the outlet of the second sensor chamber and opening of the third sensor chamber includes a sixth vertical turn along the horizontal axis, a second horizontal turn along the horizontal axis, and a seventh vertical turn along the vertical axis subsequent to the second horizontal turn.

20. The gas sensor module of claim 18, wherein the third sensor chamber is a humidity and/or temperature sensor chamber and third sensor is a humidity and/or temperature sensor.
